(19) United States
(12) Patent Application Publication
(10) Pub. No.: US 2026/0296995 A1
(43) Pub. Date: Oct. 1, 2026

(54) METHODS FOR PRODUCING 1,1-DIFLUOROPROPENE (HFO-1252ZC) FROM 1-CHLORO-3,3,3-TRIFLUOROPROPENE (HCFO-1233ZD)

(71) Applicant: Solstice Advanced Materials US, Inc., Morris Plains, NJ (US)

(72) Inventors: Selma Bektesevic, Williamsville, NY (US); Joshua DeMuth, Charlotte, NC (US); Akbar Mahdavi-Shakib, Buffalo, NY (US); Haiyou Wang, Amherst, NY (US)

(21) Appl. No.: 19/578,367

(22) Filed: Mar. 25, 2026

Related U.S. Application Data
(60) Provisional application No. 63/780,026, filed on Mar. 28, 2025.

(51) Int. Cl. C07C 17/25; C07C 17/23; C07C 17/383

(52) U.S. Cl. CPC C07C 17/25; C07C 17/23; C07C 17/383; C07C 2521/02; C07C 2523/44; C07C 2527/20; C07C 2527/24

(57) ABSTRACT

A method for producing 1,1-difluoropropene (HFO-1252zc) from 1-chloro-3,3,3-trifluoropropene (HCFO-1233zd) in the form of a two-step reaction is described. Step one of the reaction includes hydrogenation of HFO-1233zd to produce 1,1,1-trifluoropropane (HFC-263fb). Step two of the reaction includes dehydrofluorination of HFC-263fb to produce HFO-1252zc. Step one includes a catalyst, and step 2 may include a catalyst.

### CROSS-REFERENCE TO RELATED APPLICATION

This application claims priority to U.S. Provisional Application No. 63/780,026 filed Mar. 28, 2025, which is herein incorporated by reference in its entirety.

### FIELD

The present disclosure relates generally to a method for producing 1,1-difluoropropene (HFO-1252zc), and more specifically to methods for producing HFO-1252zc from 1-chloro-3,3,3-trifluoropropene (HCFO-1233zd).

### BACKGROUND

Fluorocarbon fluids have properties that are desirable for use as heat transfer media, immersion coolants, liquid or gaseous dielectrics, industrial refrigerants, and other applications.

For example, 1,1-difluoropropene (HFO-1252zc) has recently found increased utility for a variety of uses. Potential end use applications of HFO-1252zc include refrigerants, either used alone or in blends with other components, solvents for organic materials, heat transfer fluid, blowing agent, and as a chemical intermediate in the synthesis of other halogenated hydrocarbon solvents.

There is a need for developing new and useful methods for synthesizing HFO-1252zc.

### SUMMARY

The present disclosure provides a method for producing 1,1-difluoropropene (HFO-1252zc) from 1-chloro-3,3,3-trifluoropropene (HCFO-1233zd) in the form of a two-step reaction. Step one of the reaction comprises hydrogenation of HFO-1233zd to produce 1,1,1-trifluoropropane (HFC-263fb). Step two of the reaction comprises dehydrofluorination of HFC-263fb to produce HFO-1252zc. Step one comprises a catalyst, and step 2 may comprise a catalyst.

In one form thereof, the present disclosure provides a method for producing HFO-1252zc by reacting 1-chloro-3,3,3-trifluoropropene (HCFO-1233zd) with hydrogen in the presence of a catalyst to produce a first product composition comprising 1,1,1-trifluoropropane (HFC-263fb); and reacting HFC-263fb from the first product composition to produce a second product composition comprising 1,1-difluoropropene (HFO-1252zc).

In one form thereof, the second reacting step is conducted in vapor phase in the presence of a second catalyst at a second temperature. In another form thereof, the second reacting step comprises reacting HFC-263fb from the first product composition in the presence of a caustic solution to produce the second product composition.

### BRIEF DESCRIPTION OF THE DRAWINGS

FIG. 1 is a schematic process flow diagram for the conversion of 1-chloro-3,3,3-trifluoropropene (HCFO-1233zd) to 1,1-difluoropropene (HFO-1252zc).

### DETAILED DESCRIPTION

### I. Definitions

As used herein, the singular forms “a”, “an” and “the” include plural unless the context clearly dictates otherwise. Moreover, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the disclosure be limited to the specific values recited when defining a range.

As used herein, the phrase “within any range encompassing any two of these values as endpoints” or “any range using any two of the foregoing values as endpoints” literally means that any range may be selected from any two of the values listed prior to such phrase regardless of whether the values are in the lower part of the listing or in the higher part of the listing. For example, a pair of values may be selected from two lower values, two higher values, or a lower value and a higher value. For example, a range of as low as 1, 2, or 3, or as high as 8, 9, or 10 followed by this phrase encompasses ranges including 1 to 10, or 2 to 8, or 3 to 9.

As used herein, “HFO-1252zc” refers to 1,1-difluoropropene.

As used herein, “HCFO-1233zd” refers to 1-chloro-3,3,3-trifluoropropene encompassing the Z-1-chloro-3,3,3-trifluoropropene isomer, the E-1-chloro-3,3,3-trifluoropropene isomer, or a combination of the two 1-chloro-3,3,3-trifluoropropene isomers.

As used herein, “HFC-263fb” refers to 1,1,1-trifluoropropane.

As used herein, “HCFC-253 isomers” refer to isomers of chlorotrifluoropropane, including but not limited to, 3-chloro-1,1,1-trifluoropropane (HCFC-253fb) and 2-chloro-1,1,1-trifluoropropane (HCFC-253db).

As used herein, “HFO-1243zf” refers to 3,3,3-trifluoropropene.

As used herein, “HFC-272fb” refers to 1,1-difluoropropane.

As used herein, “HFC-281” refers to fluoropropane.

As used herein, the phrase “based on total moles of organic components of the composition” refers only to carbon-containing components and does not include or encompass non-carbon-containing components such as hydrogen (H₂) or hydrogen chloride (HCl).

As used herein, conversion of a reactant molecule (molecule X) during a reaction is calculated using the following equation when substantially pure reactant is used:

$$\% conversion of molecule X = ( 100 - molecule X mol . \% in the organic components of a product mixture )$$

When the reactant includes impurities or recycled components, i.e., is a component in a reaction mixture, the conversion of a reactant molecule (molecule X) during a reaction is calculated using the following equations:

$$\% conversion of molecule X = ( change in X mol . \% ) / ( X mol \% in the reactant mixture ) \\ OR \\ \% conversion of molecule X = ( X mol \% in the reactant mixture - X mol \% in the product mixture ) / ( X mol \% in the reactant mixture )$$

As used herein, selectivity to a molecule formed during a reaction (molecule X) is calculated using the following equation:

$$\% selectivity to molecule X = mol . \% of molecule X in the organic components of a product mixture / ( 100 - mol . \% of reactant molecules in the organic components of a \\ product mixture ) \times 100.$$

### II. Overview

The present disclosure provides a method for producing 1,1-difluoropropene (HFO-1252zc) from 1-chloro-3,3,3-trifluoropropene (HCFO-1233zd) according to a two-step process shown below (“Process 1”), which includes the following two steps: (i) hydrogenating HFO-1233zd to produce HFC-263fb, and (ii) dehydrofluorinating HFC-263fb to produce HFO-1252zc.

Schematic equations for the two steps (“Step (i)”, “Step (ii) Vapor Phase”, and “Step (ii) Liquid Phase”) of Process 1 are represented below:

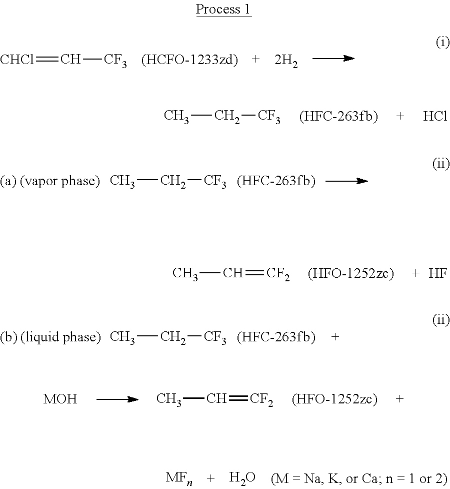

Step (i) of Process 1 proceeds through a vapor phase reaction, whereas Step (ii) of Process 1 may proceed through either a vapor phase reaction or a liquid phase reaction. Further details regarding each of Steps (i), (ii)-(a), and (ii)-(b) are set forth below.

### III. Process Flow

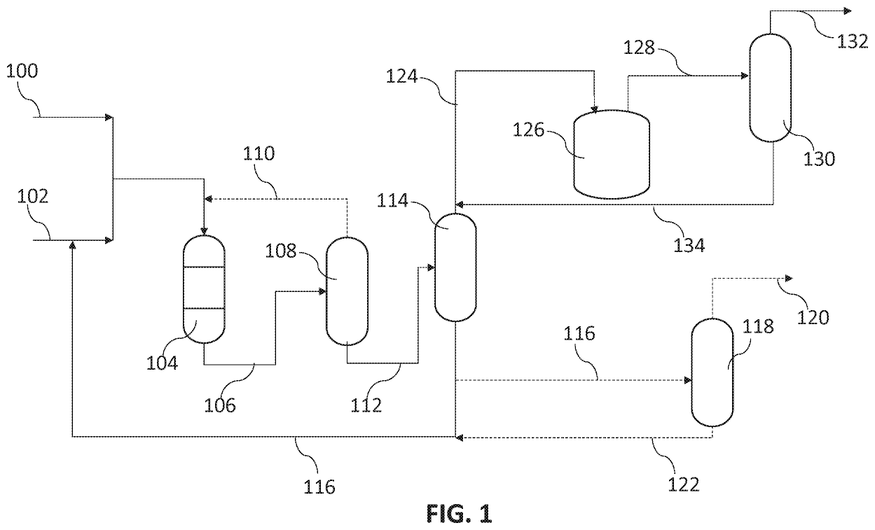

FIG. 1 is a schematic process flow diagram for the conversion of 1-chloro-3,3,3-trifluoropropene (HCFO-1233zd) to 1,1-difluoropropene (HFO-1252zc).

Referring to FIG. 1, stream 100 comprising hydrogen and stream 102 comprising HFO-1233zd enters reactor 104. Inside reactor 104, HFO-1233zd undergoes hydrogenation reaction of Step (i) to produce a first product composition comprising CH₃—CH₂—CF₃ (HFC-263fb). Stream 106 including first product composition enters first distillation column 108 to remove lights from the product stream. The top stream 110 from first distillation column 108 containing lights (e.g., propane, HFO-1243zf) may be recycled to enter reactor 104 for further hydrogenation after removing propane from stream 110. Bottom stream 112 from first distillation column 108 comprising HFC-263fb, HFC-272fb, HFC-281, and HCFC-253 isomers (HCFC-253db and HCFC-253fb) and unreacted HCFO-1233zd (if any) enters second distillation column 114.

Bottom stream 116 from second distillation column 114 comprising HFC-272fb, HCFC-253 isomers, and unreacted HCFO-1233zd (if any) may either be directly recycled back to reactor 104 or may optionally enter third distillation column 118 to remove HFC-272fb from the stream 116. Top stream 120 containing HFC-272fb from third distillation column 118 may be discarded while bottom stream 122 containing recyclable intermediates (e.g., HCFC-253 isomers) may optionally be sent back to reactor 104 for conversion to HFC-263fb via hydrogenation reaction.

Top stream 124 from second distillation column 114 comprising HFC-263fb and HFC-281 enters second reactor 126 where HFC-263fb undergoes Step (ii) dehydrofluorination reaction to produce HFO-1252zc. The effluent stream 128 from second reactor 126 comprising HFO-1252zc, HFC-263fb, and HFC-281 enters fourth distillation column 130 for the separation of desired product HFO-1252zc product from unreacted HFC-263fb. Top stream 132 from fourth distillation column 130 contains about 99.9% of desired product HFC-1252zc. Bottom stream 134 from fourth distillation column 130 containing HFC-263fb and HFC-281 is collected or sent back to second reactor 126. Occasional/optional venting may be performed on stream 134 to prevent HFC-281 accumulation in the system.

The hydrogenation reaction of Step (i) of Process 1 is carried out in the gas or vapor phase in a suitable reactor, for example a tubular reactor made from a material which is resistant to temperature and/or corrosion such as stainless steel, as well as nickel and its alloys, including Hastelloy (for example, Hastelloy C276), Inconel (for example, Inconel 600), Incoloy, and Monel, and the vessels may be lined with fluoropolymers.

The vapor phase reactors may be first cleaned and flushed with an inert gas such as nitrogen, followed by packing with a catalyst such as those described below. The catalyst may be pretreated within the reactor such as by drying in the manner described further below, followed by metering the reactants into the reactor to initiate the reaction.

The process flow for the Step (i) vapor phase hydrogenation reaction may be in the down or up direction through reactor 104. Products may be flowed through one or more scrubbers (not shown) to remove undesired byproducts from the reaction, such as hydrogen fluoride (HF) and/or hydrogen chloride (HCl), and the reaction products may be collected by capture in a cooled cylinder, for example.

The dehydrofluorination reaction of Step (ii) may be carried out in vapor phase in a suitable vapor phase reactor similar to reactor 104 and as described above. Alternatively, the dehydrofluorination reaction of Step (ii) may be carried out in liquid phase in a suitable liquid phase reactor, for example a reactor (e.g., a Parr® reactor) made from a material which is resistant to temperature, pressure and/or corrosion such as stainless steel, nickel and its alloys, including Hastelloy (for example, Hastelloy C276), Inconel (for example Inconel 600), Incoloy, and Monel wherein the vessels which may be lined with fluoropolymers.

The liquid-phase reactor may be first cleaned and flushed with deionized water, followed by charging with a basic solution made from metal hydroxides (MOH), for example, alkali metal hydroxides (e.g., LiOH, NaOH, KOH) or alkaline earth metal hydroxides (e.g., Mg(OH)₂, Ca(OH), Sr(OH)₂), or mixtures thereof.

The reaction conditions for each of Step (i) and Step (ii) in Process 1 are discussed in more details below.

### IV. Step (i)

As discussed above, Step (i) of Process 1 includes hydrogenating HFO-1233zd to produce HFC-263fb. The feed (e.g., reactant composition) for Step (i) includes HFO-1233zd (e.g., HFO-1233zdE, HFO-1233zdZ, or a mixture of HFO-1233zdE and HFO-1233zdZ). The catalyst and process conditions used in Step (i) hydrogenation reaction of Process 1 is detailed below.

### Step (i) Catalyst

In the hydrogenation reaction of Step (i), the catalyst may comprise a metal such as palladium, platinum, or nickel. The catalyst active to catalyze the reaction may preferably be palladium metal (Pd), platinum metal (Pt), or a combination of palladium metal and platinum metal.

### Step (i) Catalyst Support

In the hydrogenation reaction of Step (i), the catalyst may be supported on a suitable support, such as carbon or alumina (aluminum oxide—Al₂O₃). The carbon may be activated carbon. The alumina may be alpha(α) alumina, theta(θ) alumina, delta(δ) alumina, or gamma(γ) alumina. The supported catalyst may be produced by impregnation of any of the suitable supports with a solution of a compound of the desired metal constituent. The support may also be in the form of pellets. After the impregnation step, the solvent may be removed using heat or under vacuum resulting in a solid mass which can be further dried and reduced to form active metal catalyst.

In the hydrogenation reaction of Step (i), the catalyst may preferably be palladium on a carbon support, or palladium on an alpha alumina support.

In the hydrogenation reaction of Step (i), the metal catalysts supported on various catalyst supports are listed in Table 1 below.

TABLE 1
Supported Metal Catalysts - Step (i) Hydrogenation Reaction

| Catalyst | Support |
|---|---|
| Pd | Activated Carbon |
| Pd | alpha(α)-Al₂O₃ |
| Pd | theta(θ)-Al₂O₃ |
| Pd | delta(δ)-Al₂O₃ |
| Pd | gamma(γ)-Al₂O₃ |
| Pt | Activated Carbon |
| Pt | alpha(α)-Al₂O₃ |
| Pt | theta(θ)-Al₂O₃ |
| Pt | delta(δ)-Al₂O₃ |
| Pt | gamma(γ)-Al₂O₃ |
| Ni | Activated Carbon |
| Ni | alpha(α)-Al₂O₃ |
| Ni | theta(θ)-Al₂O₃ |
| Ni | delta(δ)-Al₂O₃ |
| Ni | gamma(γ)-Al₂O₃ |
| Rh | Activated Carbon |
| Rh | alpha(α)-Al₂O₃ |
| Rh | theta(θ)-Al₂O₃ |
| Rh | delta(δ)-Al₂O₃ |
| Rh | gamma(γ)-Al₂O₃ |

### Step (i) Catalyst Loading

For each catalyst/support combination (each row) in Table 1 used in the hydrogenation reaction of Step (i), the amount of metal loading on the support is from about 0.01 wt. %, about 0.05 wt. %, about 0.1 wt. %, about 0.2 wt. %, about 0.3 wt. %, about 0.4 wt. %, about 0.5 wt. %, or about 1 wt. % to about 2 wt. %, about 3 wt. %, about 5 wt. %, about 10 wt. %, about 20 wt. %, about 30 wt. %, about 40 wt. %, about 50 wt. % or within any range encompassed by two of the foregoing values as endpoints, for example, from about 0.01 wt. % to about 20 wt. %, from about 0.01 wt. % to about 10 wt. %, from about 0.1 wt. % to about 10 wt. %, 0.2 wt. % to about 10 wt. %, from about 0.2 wt. % to about 5 wt. %, or from about 0.2 wt. % to about 4 wt. %, based on a total weight of the catalyst and support. For supported noble metal catalysts such as Pd supported on alpha alumina, the metal loading may be from about 0.01 wt. % to about 0.5 wt. % based on a total weight of the catalyst and support. For supported noble metal catalysts such as Pd supported on activated carbon, the metal loading may be from about 1 wt. % to about 5 wt. % based on a total weight of the catalyst and support.

### Step (i) BET Surface Area

The BET (Brunauer, Emmet, and Teller) analysis is the standard method for determining surface areas from nitrogen adsorption isotherms. The BET surface areas of catalysts may be measured using TriStar II Micromeritics instrument. Catalyst samples are degassed using FlowPrep 060 instrument before BET analysis.

For each catalyst/support combination (each row) in Table 1 used in the hydrogenation reaction of Step (i), the BET surface area may be as low as about 0.5 m²/g, about 1 m²/g, about 3 m²/g, about 5 m²/g, about 10 m²/g, about 15 m²/g, about 20 m²/g², about 30 m²/g, about 40 m²/g, about 50 m²/g, about 100 m²/g, about 200 m²/g, or as high as about 250 m²/g, about 300 m²/g, about 400 m²/g, about 500 m²/g, about 600 m²/g, about 700 m²/g m², about 800 m²/g, about 900 m²/g, about 1000 m²/g, about 2000 m²/g, about 3000 m²/g, or within any range encompassed by any of the foregoing values as endpoints, from example, from about 0.5 m²/g to about 3000 m²/g, from about 1 m²/g to about 2000 m²/g, from about 1000 m²/g to about 2000 m²/g, from about 0.5 m²/g to about 500 m²/g, from about 0.5 m²/g to about 300 m²/g, from about 0.5 m²/g to about 0 m²/g, or from about 200 m²/g to about 300 m²/g.

For carbon supported metal catalysts (Pd, Pt, Ni) used in the hydrogenation reaction of Step (i), the BET surface area may be from about 100 m²/g to about 3000 m²/g, preferably from about 200 m²/g to about 2000 m²/g, more preferably from about 500 m²/g to about 1500 m²/g, and most preferably from about 1000 m²/g to about 1500 m²/g. Specific examples of additional suitable ranges are set forth below in Table 2. The numerical ranges set forth in Table 2 below are understood to be prefaced by “about”.

TABLE 2
BET Surface Area - Step (i) Hydrogenation Reaction

| From (m²/g) | To (m²/g) |
|---|---|
| 0.5 | 3000 |
| 0.5 | 2000 |
| 0.5 | 1500 |
| 0.5 | 1000 |
| 1 | 3000 |
| 1 | 2000 |
| 1 | 1500 |
| 1 | 1000 |
| 10 | 3000 |
| 10 | 2000 |
| 10 | 1500 |
| 10 | 1000 |
| 100 | 3000 |
| 100 | 2000 |
| 100 | 1500 |
| 100 | 1000 |
| 200 | 3000 |
| 200 | 2000 |
| 200 | 1500 |
| 200 | 1000 |
| 500 | 3000 |
| 500 | 2000 |
| 500 | 1500 |
| 500 | 1000 |
| 1000 | 3000 |
| 1000 | 2000 |
| 1000 | 1500 |

For alumina (alpha(α)-Al₂O₃, theta(θ)-Al₂O₃, delta(δ)-Al₂O₃, or gamma(γ)-Al₂O₃) supported metal catalysts (Pd, Pt, Ni) used in the hydrogenation reaction of Step (i), the BET surface area may be from about 0.5 m²/g to about 500 m²/g, preferably from about 1 m²/g to about 200 m²/g, more preferably from about 1 m²/g to about 100 m²/g, and most preferably from about 1 m²/g to about 20 m²/g.

### Step (i) Catalyst Pretreatment

For each catalyst/support combination (each row) in Table 1 used in the hydrogenation reaction of Step (i), the catalyst may be pretreated by a variety of methods to improve its performance and effectiveness in the reaction. For example, the catalyst may be dried at elevated temperatures, as low as about 100° C., as low as about 150° C. as low as about 200° C., about 250° C., about 300° C., about 350° C., about 360° C., about 370° C., or as high as about 380° C., about 390° C., about 400° C., about 450° C., about 500° C., about 600° C., about 700° C., or within any range encompassed by two of the foregoing values as endpoints, such as from about 100° C. to about 400° C., from about 200° C. to about 350° C., from about 250° C. to about 350° C., from about 250° C. to about 300° C., or from about 260° C. to about 300° C.

For each catalyst/support combination (each row) in Table 1 used in the hydrogenation reaction of Step (i), as part of the catalyst pretreatment, the catalyst (Pd, Pt, Rh, Ni) may be exposed to a gas such as H₂. The pretreatment process may take as low as about 1 hour, about 2 hours, about 3 hours, about 4 hours, or as high about 5 hours, about 6 hours, about 10 hours, about 20 hours, or within any range encompassed by two of the foregoing values as endpoints, for example from about 1 hour to about 20 hours, from about 2 hours to about 10 hours, from about 5 hours to about 10 hours, or from about 7 hours to about 9 hours.

### Step (i) Reaction Temperature

For reactions using each catalyst/support combination (each row) in Table 1, the reaction temperature of the hydrogenation reaction of Step (i) may be as low as about 100° C., about 125° C., about 150° C., about 200° C., or as high as about 250° C., about 275° C., about 300° C., about 350° C., about 400° C., or within any range encompassed by two of the foregoing values as endpoints, such as from about 100° C. to about 400° C., or from about 125° C. to about 350° C., for example. The temperature may be preferably from about 100° C. to about 300° C., and more preferably from about 150° C. to about 275° C. Specific examples of additional suitable ranges are set forth below in Table 3. The numerical ranges set forth in Table 3 below are understood to be prefaced by “about”.

TABLE 3
Temperature Ranges - Step (i) Hydrogenation Reaction

| From (° C.) | To (° C.) |
|---|---|
| 100 | 400 |
| 100 | 350 |
| 100 | 300 |
| 100 | 275 |
| 100 | 250 |
| 100 | 200 |
| 100 | 150 |
| 125 | 400 |
| 125 | 350 |
| 125 | 300 |
| 125 | 275 |
| 125 | 250 |
| 125 | 200 |
| 125 | 150 |
| 150 | 400 |
| 150 | 350 |
| 150 | 300 |
| 150 | 275 |
| 150 | 250 |
| 150 | 200 |
| 200 | 400 |
| 200 | 350 |
| 200 | 300 |
| 200 | 275 |
| 200 | 250 |

### Step (i) Reaction Contact Time

For reactions using each catalyst/support combination (each row) in table 1, the contact time of the reactants with the catalyst (Pd, Pt, Rh, Ni) in the hydrogenation reaction of Step (i) may be as little as about 0.1 second, about 1 second, about 2 seconds, about 5 seconds, about 10 seconds, about 15 seconds, about 20 seconds, or as long as about 25 seconds, about 30 seconds, about 40 seconds, about 50 seconds, about 60 seconds, about 80 seconds, about 120 seconds, or within any range encompassed by two of the foregoing values as endpoints, such as from about 0.1 seconds to about 120 seconds, from about 1 second to about 60 seconds, from about 5 seconds to about 50 seconds, from about 10 seconds to about 40 seconds, from about 15 seconds to about 30 seconds, or about 20 seconds to about 25 seconds. For example, the contact time may be from about 1 second to about 60 seconds.

### Step (i) Reaction Pressure

For reactions using each catalyst/support combination (each row) in Table 1, the pressure inside which reactor the hydrogenation reaction of Step (i) takes place may be as little as about 1 psig, about 3 psig, about 5 psig, about 10 psig, about 15 psig, about 20 psig, about 30 psig, about 35 psig or about 40 psig, or as great as about 90 psig, about 100 psig, about 120 psig, about 150 psig, about 200 psig or about 250 psig, about 300 psig, or within any range encompassed by two of the foregoing values as endpoints, such as from about 1 psig to about 300 psig, from about 3 psig to about 250 psig, from about 5 psig to about 200 psig, from about 10 psig to about 150 psig, from about 15 psig to about 120 psig, from about 20 psig to about 100 psig, from about 30 psig to about 90 psig, or from about 35 psig to about 50 psig. For example, the pressure may be from about 10 psig to about 200 psig. Specific examples of additional suitable ranges are set forth below in Table 4. The numerical ranges set forth in Table 4 below are understood to be prefaced by “about”.

TABLE 4
Pressure Ranges - Step (i) Hydrogenation Reaction

| From (psig) | To (psig) |
|---|---|
| 1 | 300 |
| 1 | 250 |
| 1 | 200 |
| 1 | 150 |
| 1 | 120 |
| 1 | 100 |
| 1 | 90 |
| 1 | 50 |
| 10 | 300 |
| 10 | 250 |
| 10 | 200 |
| 10 | 150 |
| 10 | 120 |
| 10 | 100 |
| 10 | 90 |
| 10 | 50 |
| 15 | 300 |
| 15 | 250 |
| 15 | 200 |
| 15 | 150 |
| 15 | 120 |
| 15 | 100 |
| 15 | 90 |
| 15 | 50 |
| 20 | 300 |
| 20 | 250 |
| 20 | 200 |
| 20 | 150 |
| 20 | 120 |
| 20 | 100 |
| 20 | 90 |
| 20 | 50 |

### Step (i) Reaction Conditions

A summary of the preferred catalyst, catalyst support, temperatures, and pressures as discussed above are summarized in Table 5 below. The numerical ranges set forth in Table 5 are understood to be prefaced by “about”.

TABLE 5
Summary of Preferred Step (i) Reaction Conditions

| Catalyst | Support | Temperature (° C.) | Pressure (psig) |
|---|---|---|---|
| Pd | alpha(α)-Al₂O₃ | 100-400 | 1-300 |
| Pd | alpha(α)-Al₂O₃ | 100-400 | 10-200 |
| Pd | alpha(α)-Al₂O₃ | 100-400 | 20-100 |
| Pd | alpha(α)-Al₂O₃ | 100-400 | 20-50 |
| Pd | alpha(α)-Al₂O₃ | 100-300 | 1-300 |
| Pd | alpha(α)-Al₂O₃ | 100-300 | 10-200 |
| Pd | alpha(α)-Al₂O₃ | 100-300 | 20-100 |
| Pd | alpha(α)-Al₂O₃ | 100-300 | 20-50 |
| Pd | alpha(α)-Al₂O₃ | 100-200 | 1-300 |
| Pd | alpha(α)-Al₂O₃ | 100-200 | 10-200 |
| Pd | alpha(α)-Al₂O₃ | 100-200 | 20-100 |
| Pd | alpha(α)-Al₂O₃ | 100-200 | 20-50 |
| Pd | alpha(α)-Al₂O₃ | 125-200 | 1-300 |
| Pd | alpha(α)-Al₂O₃ | 125-200 | 10-200 |
| Pd | alpha(α)-Al₂O₃ | 125-200 | 20-100 |
| Pd | alpha(α)-Al₂O₃ | 125-200 | 20-50 |
| Pd | Activated Carbon | 100-400 | 1-300 |
| Pd | Activated Carbon | 100-400 | 10-200 |
| Pd | Activated Carbon | 100-400 | 20-100 |
| Pd | Activated Carbon | 100-400 | 20-50 |
| Pd | Activated Carbon | 100-300 | 1-300 |
| Pd | Activated Carbon | 100-300 | 10-200 |
| Pd | Activated Carbon | 100-300 | 20-100 |
| Pd | Activated Carbon | 100-300 | 20-50 |
| Pd | Activated Carbon | 150-300 | 1-300 |
| Pd | Activated Carbon | 150-300 | 10-200 |
| Pd | Activated Carbon | 150-300 | 20-100 |
| Pd | Activated Carbon | 150-300 | 20-50 |
| Pd | Activated Carbon | 200-300 | 1-300 |
| Pd | Activated Carbon | 200-300 | 10-200 |
| Pd | Activated Carbon | 200-300 | 20-100 |
| Pd | Activated Carbon | 200-300 | 20-50 |
| Pd | Activated Carbon | 250-300 | 1-300 |
| Pd | Activated Carbon | 250-300 | 10-200 |
| Pd | Activated Carbon | 250-300 | 20-100 |
| Pd | Activated Carbon | 250-300 | 20-50 |

### Step (i) Reaction Hydrogen Mole Ratio

For reactions using each catalyst/support combination (each row) in Table 1 used in the hydrogenation reaction of Step (i), the mole ratio of hydrogen to HCFO-1233zd may be as little about 1:1, about 2:1, about 3:1, about 4:1, about 5:1, about 5.5:1 or as great as about 6:1, about 6.5:1, about 7.5:1 or about 8:1, about 10:1, for example, or within any range encompassed by two of the foregoing values as endpoints. The mole ratio of hydrogen to HCFO-1233zd may be preferably from about 2:1 about to 15:1, and more preferably from about 3:1 to about 5:1.

### Step (i) Conversion of the Starting Material

As demonstrated by the Examples herein, for reactions using each catalyst/support combination (each row) in Table 1, the hydrogenation Step (i) may achieve a conversion of the starting material of greater than about 70%, greater than about 80%, greater than about 90%, greater than about 95%, greater than about 99%, and for each of the foregoing, less than or equal to 100%, or within any range encompassed by two of the foregoing values as endpoints, such as from about 70% to about 100%, from about 95% to about 100%, or from about 99% to about 100%.

### Step (i) Selectivity to HFC-263fb

As demonstrated by the Examples herein, for reactions using each catalyst/support combination (each row) in Table 1, the hydrogenation Step (i) may achieve a selectivity to the desired product HFC-263fb product of at least about 20%, at least about 30%, at least about 40%, at least about 50%, at least about 60%, at least about 70%, at least about 80%, at least about 90%, at least about 95%, at least about 99%, and for each of the foregoing, less than or equal to 100%, or within any range encompassed by two of the foregoing values as endpoints, such as from about 20% to about 99%, from about 30% to about 95%, from about 40% to about 95%, or about 50% to about 95%, based on total moles of the organic components of the composition.

### Step (i) Product Composition

For a hydrogenation reaction of Step (i) carried out using each catalyst/support combination (each row) in Table 1, referring to FIG. 1, the amount of HFC-263fb in the product composition in stream 106 from the reactor 104 may be at least 70 mol %, at least 80 mol %, or at least 90 mol %, and for each of the foregoing, less than or equal to 100 mol %, for example, based on total moles of organic components of the composition.

For a hydrogenation reaction of Step (i) carried out using each catalyst/support combination (each row) in Table 1, referring to FIG. 1, the amount of HCFC-253 isomers in the product composition in stream 106 from the reactor 104, if present, may be greater than or equal to 0.01 mol % and yet less than 15 mol %, less than 10 mol %, or less than 5 mol %, for example, based on total moles of organic components of the composition.

For a hydrogenation reaction of Step (i) carried out using each catalyst/support combination (each row) in Table 1, referring to FIG. 1, the amount of HFO-1243zf in the product composition in stream 106 from the reactor 104, if present, may be greater than or equal to 0.01 mol % and yet less than 5 mol %, less than 4 mol %, less than 3 mol %, less than 2 mol %, or less than 1 mol %, for example, based on total moles of organic components of the composition.

For a hydrogenation reaction of Step (i) carried out using each catalyst/support combination (each row) in Table 1, referring to FIG. 1, the amount of HFC-272fb in the product composition in stream 106 from the reactor 104, if present, may be greater than or equal to 0.01 mol % and yet less than 5 mol %, less than 4 mol %, less than 3 mol %, or less than 2 mol %, for example, based on total moles of organic components of the composition.

For a hydrogenation reaction of Step (i) carried out using each catalyst/support combination (each row) in Table 1, referring to FIG. 1, the total amount of HFC-263fb, HCFC-253 isomers, and HFO-1243zf in the product composition in stream 106 from the reactor 104 may be at least 70 mol %, at least 80 mol %, at least 85 mol %, at least 90 mol %, at least 99 mol %, or at least 99 mol %, and for each of the foregoing, less than or equal to 100 mol %, for example, based on total moles of organic components of the composition.

For example, for a hydrogenation reaction of Step (i) carried out using each catalyst/support combination (each row) in Table 1, referring to FIG. 1, in the product composition in stream 106 from the reactor 104, the amount of HFC-263fb may be at least 85 mol % and less than or equal to 99.8 mol %, the amount of HCFC-253 isomers may be at least 0.01 mol % and less than or equal to 10 mol %, the amount of HFO-1243zf may be at least 0.01 mol % and less than or equal to 5 mol %, based on the combined total moles of the HFC-263fb, HCFC-253 isomers, and HFO-1243zf in the product composition.

The combined amount of HFC-263fb, HCFC-253 isomers, and HFO-1243zf in the composition set forth in the preceding paragraph may be at least 85 mol % and less than or equal to 100% of the total moles of organic components of the product composition in stream 106 from the reactor 104, while the combined amount of other components including undesired byproducts (e.g., HFC-272fb) may be greater than or equal to 0 mol % and less than 15 mol % of the total moles of organic components of the product composition in stream 106 from the reactor 104.

For example, for a hydrogenation reaction of Step (i) carried out using each catalyst/support combination (each row) in Table 1, referring to FIG. 1, in the product composition in stream 106 from the reactor 104, the amount of HFC-263fb may be at least 90 mol % and less than or equal to 99.8 mol %, the amount of HCFC-253 isomers may be at least 0.01 mol % and less than or equal to 8 mol %, the amount of HFO-1243zf may be at least 0.01 mol % and less than or equal to 2 mol %, based on the combined total moles of the HFC-263fb, HCFC-253 isomers, and HFO-1243zf in the product composition.

The combined amount of HFC-263fb, HCFC-253 isomers, and HFO-1243zf in the composition set forth in the preceding paragraph may be at least 90 mol % and less than or equal to 100% of the total moles of organic components of the product composition in stream 106 from the reactor 104, while the combined amount of other components including undesired byproducts (e.g., HFC-272fb) may be greater than or equal to 0 mol % and less than 10 mol % of the total moles of organic components of the product composition in stream 106 from the reactor 104.

### Step (i) Post Processing

Referring again to FIG. 1, the product stream 106 from reactor 104 may go through one or more post processing steps for separating HFC-263fb with recyclable intermediates (e.g., HCFC-253 isomers, and HFO-1243zf) and/or undesired byproducts (e.g., HFC-272fb) before proceeding to Step (ii) of Process 1. For example, product stream 106 may go through a first distillation column 108 for separating recyclable intermediate HFO-1243zf from the product stream 106, then a second distillation column 114 to produce a composition with higher purity of HFC-263fb to serve as feed stream for Step (ii) of Process 1. The Bottom stream 116 from second distillation column 114 may optionally enter a third distillation column 118 for separating recyclable intermediate HCFC-253 isomers from undesired byproducts.

### Step (i) Top Stream 110 Composition

For a hydrogenation reaction of Step (i) carried out using each catalyst/support combination (each row) in Table 1, referring to FIG. 1, the amount of HFO-1243zf in top stream 110 may be greater than 15 mol %, greater than 20 mol %, greater than 25 mol %, or greater than 30 mol %, and for each of the foregoing, less than or equal to 100 mol %, for example, based on total moles of organic components in the composition.

For a hydrogenation reaction of Step (i) carried out using each catalyst/support combination (each row) in Table 1, referring to FIG. 1, the amount of HFO-1243zf in top stream 110 may be less than 80 mol %, less than 75 mol %, or less than 70 mol %, and for each of the foregoing, greater than or equal to 0.01 mol %, for example, based on total moles of organic components of the composition.

Top stream 110 may optionally be recycled back to reactor 104 for HFO-1243zf hydrogenation after occasional vent to remove propane from the top stream 110.

### Step (i) Bottom Stream 112 Composition

For a hydrogenation reaction of Step (i) carried out using each catalyst/support combination (each row) in Table 1, referring to FIG. 1, the amount of HFC-263fb in bottom stream 112 may be greater than 80 mol %, greater than 85 mol %, greater than 90 mol %, or greater than 95 mol %, and for each of the foregoing, less than or equal to 100 mol %, for example, based on total moles of organic components in the composition.

For a hydrogenation reaction of Step (i) carried out using each catalyst/support combination (each row) in Table 1, referring to FIG. 1, the amount of HFC-272fb in bottom stream 112, if present, may be less than 10 mol %, less than 5 mol %, or less than 3 mol %, and for each of the foregoing, greater than or equal to 0.01 mol %, for example, based on total moles of organic components of the composition.

For a hydrogenation reaction of Step (i) carried out using each catalyst/support combination (each row) in Table 1, referring to FIG. 1, the amount of HFC-281 in bottom stream 112, if present, may be less than 5 mol %, less than 2 mol %, or less than 1 mol %, and for each of the foregoing, greater than or equal to 0.01 mol %, for example, based on total moles of organic components of the composition.

For a hydrogenation reaction of Step (i) carried out using each catalyst/support combination (each row) in Table 1, referring to FIG. 1, the amount of HCFC-253 isomers in bottom stream 112, if present, may be less than 10 mol %, less than 5 mol %, or less than 3 mol %, and for each of the foregoing, greater than or equal to 0.01 mol %, for example, based on total moles of organic components of the composition.

For a hydrogenation reaction of Step (i) carried out using each catalyst/support combination (each row) in Table 1, referring to FIG. 1, the amount of unreacted HCFO-1233zd in bottom stream 112, if present, may be less than 10 mol %, less than 5 mol %, or less than 3 mol %, and for each of the foregoing, greater than or equal to 0.01 mol %, for example, based on total moles of organic components of the composition.

### Step (i) Bottom Stream 116 Composition and Processing

For a hydrogenation reaction of Step (i) carried out using each catalyst/support combination (each row) in Table 1, referring to FIG. 1, the amount of HCFC-253 isomers in bottom stream 116 may be greater than 15 mol %, greater than 20 mol %, greater than 25 mol %, or greater than 30 mol %, and for each of the foregoing, less than or equal to 100 mol %, for example, based on total moles of organic components in the composition.

For a hydrogenation reaction of Step (i) carried out using each catalyst/support combination (each row) in Table 1, referring to FIG. 1, the amount of HFC-281 in bottom stream 112, if present, may be less than 5 mol %, less than 2 mol %, or less than 1 mol %, and for each of the foregoing, greater than or equal to 0.01 mol %, for example, based on total moles of organic components of the composition.

For a hydrogenation reaction of Step (i) carried out using each catalyst/support combination (each row) in Table 1, referring to FIG. 1, the amount of HFC-272fb in bottom stream 116 may be less than 80 mol %, less than 75 mol %, or less than 70 mol %, and for each of the foregoing, greater than or equal to 0.01 mol %, for example, based on total moles of organic components of the composition.

For a hydrogenation reaction of Step (i) carried out using each catalyst/support combination (each row) in Table 1, referring to FIG. 1, the amount of unreacted HCFO-1233zd in bottom stream 112, if present, may be less than 10 mol %, less than 5 mol %, or less than 3 mol %, and for each of the foregoing, greater than or equal to 0.01 mol %, for example, based on total moles of organic components of the composition.

Bottom stream 116 may optionally enter a third distillation column 118 for producing a recycle stream 122 containing substantially pure HCFC-253 isomers for recycling back to reactor 104 for hydrogenation and conversion to HFC-263fb.

### Step (i) Top Stream 124 Composition

For a hydrogenation reaction of Step (i) carried out using each catalyst/support combination (each row) in Table 1, referring to FIG. 1, the amount of HFC-263fb in top stream 124 may be greater than 90 mol %, greater than 95 mol %, greater than 98 mol %, or greater than 99 mol %, and for each of the foregoing, less than or equal to 100 mol %, for example, based on total moles of organic components in the composition.

For a hydrogenation reaction of Step (i) carried out using each catalyst/support combination (each row) in Table 1, referring to FIG. 1, the amount of HFC-281 in top stream 124, if present, may be less than 5 mol %, less than 2 mol %, or less than 1 mol %, and for each of the foregoing, greater than or equal to 0.01 mol %, for example, based on total moles of organic components of the composition.

Top stream 124 containing substantially pure HFC-263fb then enters second reactor 126 for dehydrofluorination reaction of Step (ii).

### V. Step (ii)-(a) Vapor Phase

As discussed above, the dehydrofluorination reaction of Step (ii) may be carried out in vapor phase in a suitable second vapor phase reactor similar to first reactor 104. Step (ii)-(a) of Process 1 includes dehydrofluorinating HFC-263fb in vapor phase to produce HFO-1252zc. The catalyst and process conditions used in Step (ii)-(a) vapor phase dehydrofluorination reaction of Process 1 is detailed below.

### Step (ii)-(a) Catalysts

Suitable catalysts for the Step (ii)-(a) vapor phase dehydrofluorination reaction include metal oxides such as chromium oxide (Cr₂O₃), zinc oxide (ZnO), nickel oxide (NiO), aluminum oxide (Al₂O₃), iron oxide (Fe₂O₃), and magnesium oxide (MgO), and various combinations of or mixtures thereof, for example, ZnO—Cr₂O₃, NiO—Cr₂O₃, and/or MgO—Cr₂O₃. Fluorination treatment of the catalyst may be conducted using anhydrous hydrogen fluoride (HF) under conditions effective to convert a portion of metal oxides into corresponding metal fluorides, such as via the procedure disclosed in U.S. Pat. No. 6,780,815 to Cerri et al., the disclosure of which is expressly incorporated by reference herein. Other suitable catalysts for the dehydrofluorination reaction include metal fluorides such as chromium fluoride (CrF₃), alumina fluoride (AlF₃), iron fluoride (FeF₃), magnesium fluoride (MgF₂), and various combinations of or mixtures thereof.

Other metals, such as Pd, Pt, and Ni, may also be loaded onto the above fluorinated metal oxides, for example, via a wet impregnation process wherein a salt of the metal is exposed to the fluorinated metal oxide support in solution, followed by drying and then reduction with hydrogen gas.

TABLE 6
Metal Catalysts - Step (ii)-(a) Vapor
Phase Dehydrofluorination Reaction

| Catalyst | Support |
|---|---|
| Pd | CrF₃ |
| Pd | AlF₃ |
| Pd | FeF₃ |
| Pd | MgF₂ |
| Pt | CrF₃ |
| Pt | AlF₃ |
| Pt | FeF₃ |
| Pt | MgF₂ |
| Ni | CrF₃ |
| Ni | AlF₃ |
| Ni | FeF₃ |
| Ni | MgF₂ |
| Cr₂O₃ | N/A |
| ZnO | N/A |
| NiO | N/A |
| Al₂O₃ | N/A |
| Fe₂O₃ | N/A |
| MgO | N/A |
| ZnO—Cr₂O₃ | N/A |
| NiO—Cr₂O₃ | N/A |
| MgO—Cr₂O₃ | N/A |
| CrF₃ | N/A |
| AlF₃ | N/A |
| FeF₃ | N/A |
| MgF₂ | N/A |

### Step (ii)-(a) Catalyst Loading (Pd, Pt, and Ni on CrF₃, AlF₃, FeF₃, or MqF₂)

For a vapor phase dehydrofluorination reaction of Step (ii)-(a) of Process 1 carried out using each catalyst/support combination (each row) in Table 6, the amount of metal loading on the support may be from about 0.01 wt. %, about 0.05 wt. %, about 0.1 wt. %, about 0.2 wt. %, about 0.3 wt. %, about 0.4 wt. %, about 0.5 wt. %, or about 1 wt. % to about 2 wt. %, about 3 wt. %, 5 wt. % 10 wt. %, or 20 wt. %, or 30 wt. %, or 40 wt. %, or 50 wt. % or within any range encompassed by two of the foregoing values as endpoints, based on a total weight of the catalyst and support, such as from about 0.01 wt. % to about 50 wt. %, from about 0.05 wt. % to about 40 wt. %, from about 0.1 wt. % to about 30 wt. %, from about 0.2 wt. % to about 20 wt. %, from about 0.3 wt. % to about 10 wt. %, from about 0.4 wt. % to about 5 wt. %, from about 0.5 wt. % to about 3 wt. %, or from about 1 wt. % to about 2 wt. %.

For supported noble metal catalysts such as platinum or palladium, the metal loading may be ranged from about 0.01 wt. % to about 5 wt. %, preferably from about 0.05 wt. % to about 2 wt. %, and more preferably from about 0.1 wt. % to about 1 wt. %. When fluorinated alumina is used, the amount of metal loading on the support may be from about 0.01 wt. % to about 5 wt. %, preferably from about 0.05 wt. % to about 2 wt. %, most preferably from about 0.1 wt. % to about 1 wt. %.

### Step (ii)-(a) Catalyst BET Surface Area

The catalyst used in Step (ii)-(a) may have a proper BET (Brunauer, Emmet, and Teller) surface area. The BET surface area of the catalyst may be as low as about 10 m²/g, about 20 m²/g, about 30 m²/g, about 40 m²/g, about 50 m²/g, about 60 m²/g, about 70 m²/g, about 80 m²/g, about 90 m²/g, about 100 m²/g, or as high as about 110 m²/g, about 120 m²/g, about 130 m²/g, about 140 m²/g, about 150 m²/g, about 175 m²/g, about 200 m²/g, about 225 m²/g, about 250 m²/g, about 300 m²/g, or within any range encompassed by any of the foregoing values as endpoints, such as from about 10 m²/g to about 300 m²/g, from about 20 m²/g to about 250 m²/g, from about 30 m²/g to about 225 m²/g, from about 40 m²/g to about 200 m²/g, from about 50 m²/g to about 175 m²/g, from about 60 m²/g to about 150 m²/g, from about 70 m²/g to about 140 m²/g, from about 80 m²/g to about 130 m²/g, from about 90 m²/g, to about 120 m²/g, or from about 100 m²/g to about 110 m²/g.

For metal oxides catalysts, the BET surface area may be preferably greater than about 100 m²/g. For fluorinated metal oxides catalysts, the BET surface area may be preferably greater than about 20 m²/g.

When fluorinated alumina is used, the BET surface area may be greater than about 10 m²/g, preferably greater than 20 m²/g, most preferably greater than 25 m²/g.

### Step (ii)-(a) Catalyst Pretreatment

The catalyst used in Step (ii)-(a) may be pretreated by drying at elevated temperatures, as low as about 100° C., about 150° C., about 200° C., about 250° C., about 300° C., about 350° C., about 360° C., about 370° C., or as high about 390° C., about 400° C., about 450° C., about 500° C., about 550° C., about 600° C., or within any range encompassed by two of the foregoing values as endpoints, such as from about 100° C. to about 600° C., from about 250° C. to about 550° C., from about 300° C. to about 500° C., from about 350° C. to about 450° C., from about 360° C. to about 400° C., or from about 370° C. to about 390° C.

When fluorinated alumina is used, the catalyst may be pretreated by drying at a temperature of from about 100° C. to about 600° C., preferably from about 300° C. to about 600° C., most preferably from about 400° C. to about 550° C.

As part of the catalyst activation, the catalyst may be exposed to an inert gas such as N₂. The pretreatment process may take as low as about 1 hour, about 2 hours, about 3 hours, about 4 hours, or as high about 5 hours, about 6 hours, about 10 hours, about 20 hours, or within any range encompassed by two of the foregoing values as endpoints such as from about 1 hour to about 20 hours, from about 2 hours to about 10 hours, from about 3 hours to about 6 hours, or from about 4 hours to about 5 hours.

When fluorinated alumina is used, the pretreatment process may take from about 1 hour to about 10 hours, preferably from about 2 hours to about 6 hours, most preferably from about 3 hours to about 5 hours.

### Step (ii)-(a) Reaction Temperature

The temperature range for dehydrofluorination reaction of Step (ii)-(a) may be as low as about 125° C., about 150° C., about 200° C., about 250° C., about 300° C., about 350° C., about 400° C., about 450° C., or as high as about 500° C., about 550° C., about 600° C., about 650° C., about 700° C., about 750° C., about 800° C. or within any range encompassed by two of the foregoing values as endpoints, such as from about 125° C. to about 800° C., from about 150° C. to about 750° C., from about 200° C. to about 650° C., from about 250° C. to about 600° C., from about 300° C. to about 550° C., about 350° C. to about 500° C., or about 400° C. to about 450° C. The temperature may be preferably from about 250° C. to about 450° C., and more preferably from about 300° C. to about 400° C.

When fluorinated alumina is used, the reaction temperature may be from about 125° C. to about 500° C., preferably from about 250° C. to about 450° C., most preferably from about 300° C. to about 400° C.

Specific examples of additional suitable ranges are set forth below in Table 7. The numerical ranges set forth in Table 7 below are understood to be prefaced by “about”.

TABLE 7
Temperature Ranges - Step (ii)-(a) Vapor
Phase Dehydrofluorination Reaction

| From (° C.) | To (° C.) |
|---|---|
| 125 | 800 |
| 125 | 700 |
| 125 | 600 |
| 125 | 500 |
| 125 | 450 |
| 125 | 400 |
| 200 | 800 |
| 200 | 700 |
| 200 | 600 |
| 200 | 500 |
| 200 | 450 |
| 200 | 400 |
| 250 | 800 |
| 250 | 700 |
| 250 | 600 |
| 250 | 500 |
| 250 | 450 |
| 250 | 400 |
| 300 | 800 |
| 300 | 700 |
| 300 | 600 |
| 300 | 500 |
| 300 | 450 |
| 300 | 400 |

### Step (ii)-(a) Reaction Pressure

The pressure for dehydrofluorination reaction of Step (ii)-(a) may be as little as about 1 psig, about 2 psig, about 3 psig, about 4 psig or about 5 psig, about 10 psig, about 15 psig, about 20 psig, about 25 psig, about 30 psig, about 35 psig, about 40 psig, about 50 psig, about 100 psig, or within any range encompassed by two of the foregoing values as endpoints, such as from about 1 psig to about 50 psig, from about 2 psig to about 40 psig, from about 3 psig to about 35 psig, from about 4 psig to about 25 psig, from about 5 psig to about 20 psig, or from about 10 psig to about 15 psig. For example, the pressure may be from about 1 psig to about 50 psig, preferably from about 5 psig to about 30 psig, and more preferably from about 10 psig to about 20 psig.

When fluorinated alumina is used, the reaction pressure may be from about 1 psig to about 100 psig, preferably from about 3 psig to about 5 psig, most preferably from about 5 psig to about 20 psig.

Specific examples of additional suitable ranges are set forth below in Table 8. The numerical ranges set forth in Table 8 below are understood to be prefaced by “about”.

TABLE 8
Pressure Ranges - Step (ii)-(a) Vapor
Phase Dehydrofluorination Reaction

| From (psig) | To (psig) |
|---|---|
| 1 | 100 |
| 1 | 50 |
| 1 | 40 |
| 1 | 30 |
| 1 | 20 |
| 1 | 10 |
| 1 | 5 |
| 3 | 100 |
| 3 | 50 |
| 3 | 40 |
| 3 | 30 |
| 3 | 20 |
| 3 | 10 |
| 3 | 5 |
| 5 | 100 |
| 5 | 50 |
| 5 | 40 |
| 5 | 30 |
| 5 | 20 |
| 5 | 10 |
| 10 | 100 |
| 10 | 50 |
| 10 | 40 |
| 10 | 30 |
| 10 | 20 |

### Step (ii)-(a) Reaction Contact Time

The contact time of the reactants with the catalyst for dehydrofluorination reaction of Step (ii)-(a) may be as little as about 0.1 second, about 1 second, about 5 seconds, about 10 seconds, about 15 seconds or about 20 seconds, or as long as about 25 seconds, about 30 seconds, about 40 seconds, about 50 seconds, about 60 seconds, about 120 seconds, about or within any range encompassed by two of the foregoing values as endpoints, such as from about 0.1 seconds to about 120 seconds, from about 1 second to about 60 seconds, from about 5 seconds to about 50 seconds, from about 10 seconds to about 40 seconds, from about 15 seconds to about 30 seconds, or from about 20 seconds to about 25 seconds. For example, the contact time may be from about 1 second to about 60 seconds.

When fluorinated alumina is used, the contact time may be from about 1 second to about 60 seconds, preferably from about 5 seconds to about 40 seconds, most preferably from about 10 seconds to about 30 seconds.

### Step (ii)-(a) Conversion of the Starting Material

The conversion of the starting material for dehydrofluorination reaction of Step (ii)-(a) may be greater than about 10%, greater than about 20%, greater than about 30%, greater than about 40%, greater than about 50%, greater than about 60%, greater than about 70%, greater than about 80%, greater than about 90%, greater than about 95%, greater than about 99%, or within any range encompassed by two of the foregoing values as endpoints, such as from about 10% to about 99%, from about 20% to about 95%, from about 30% to about 80%, from about 40% to about 70%, or from about 50% to about 60%.

When fluorinated alumina is used, the conversion of the starting material to 1,2-difluoroethylene may be greater than about 20%, preferably greater than about 30%, most preferably greater than about 60%.

### Step (ii)-(a) Selectivity to HFO-1252zc

The selectivity to the desired product HFO-1252zc for dehydrofluorination reaction of Step (ii)-(a) may be as low as about 80%, about 85%, about 89% about 90%, about 91%, about 92%, or as high as about 94%, about 95% about 96%, about 97%, about 98%, about 99%, or within any range encompassed by two of the foregoing values as endpoints, such as from about 80% to about 99%, from about 85% to about 98%, from about 89% to about 97%, from about 90% to about 96%, from about 91% to about 95%, or from about 92% to about 94%.

When fluorinated alumina is used, the selectivity to the desired 1,2-difluoroethylene product may be from about 85% to about 99%, preferably from about 90% to about 99%, most preferably from about 95% to about 99%.

### Step (ii)-(a) Product Composition

For a dehydrofluorination reaction of Step (ii)-(a) carried out using each catalyst/support combination (each row) in Table 6, referring to FIG. 1, the amount of HFO-1252zc in the product composition in stream 128 from the second reactor 126 may be at least 15 mol %, at least 25 mol %, at least 30 mol %, or at least 35 mol %, and for each of the foregoing, less than or equal to 100 mol %, for example, based on total moles of organic components of the composition.

For a dehydrofluorination reaction of Step (ii)-(a) carried out using each catalyst/support combination (each row) in Table 6, referring to FIG. 1, the amount of unreacted HFC-263fb in the product composition in stream 128 from the second reactor 126 may be greater than or equal to 0.1 mol % and yet less than 85 mol %, less than 75 mol %, less than 70 mol %, or less than 65 mol %, for example, based on total moles of organic components of the composition.

For a dehydrofluorination reaction of Step (ii)-(a) carried out using each catalyst/support combination (each row) in Table 6, referring to FIG. 1, the amount of undesired byproduct HFC-281 in the product composition in stream 128 from the second reactor 126, if present, may be greater than or equal to 0.1 mol % and yet less than 5 mol %, less than 4 mol %, or less than 2 mol %, for example, based on total moles of organic components of the composition.

For example, for a dehydrofluorination reaction of Step (ii)-(a) carried out using each catalyst/support combination (each row) in Table 6, referring to FIG. 1, in the product composition in stream 128 from the second reactor 126, the amount of HFO-1252zc may be at least 15 mol % and less than or equal to 99.9 mol %, the amount of HFC-263fb may be at least 0.1 mol % and less than or equal to 85 mol %, based on the combined total moles of the HFO-1252zc and HFC-263fb in the product composition.

The combined amount of HFO-1252zc and HFC-263fb in the composition set forth in the preceding paragraph may be at least 90 mol % and less than or equal to 100% of the total moles of organic components of the product composition in stream 128 from the second reactor 126, while the combined amount of other components including undesired byproducts (e.g., HFC-281) may be greater than or equal to 0 mol % and less than 10 mol % of the total moles of organic components of the product composition in stream 128 from the second reactor 126.

For example, for a dehydrofluorination reaction of Step (ii)-(a) carried out using each catalyst/support combination (each row) in Table 6, referring to FIG. 1, in the product composition in stream 128 from the second reactor 126, the amount of HFO-1252zc may be at least 30 mol % and less than or equal to 99.9 mol %, the amount of HFC-263fb may be at least 0.1 mol % and less than or equal to 70 mol %, based on the combined total moles of the HFO-1252zc and HFC-263fb in the product composition.

The combined amount of HFO-1252zc and HFC-263fb in the composition set forth in the preceding paragraph may be at least 95 mol % and less than or equal to 100% of the total moles of organic components of the product composition in stream 128 from the second reactor 126, while the combined amount of other components including undesired byproducts (e.g., HFC-281) may be greater than or equal to 0 mol % and less than 5 mol % of the total moles of organic components of the product composition in stream 128 from the second reactor 126.

For example, for a dehydrofluorination reaction of Step (ii)-(a) carried out using each catalyst/support combination (each row) in Table 6, referring to FIG. 1, in the product composition in stream 128 from the second reactor 126, the amount of HFO-1252zc may be at least 35 mol % and less than or equal to 99.9 mol %, the amount of HFC-263fb may be at least 0.1 mol % and less than or equal to 65 mol %, based on the combined total moles of the HFO-1252zc and HFC-263fb in the product composition.

The combined amount of HFO-1252zc and HFC-263fb in the composition set forth in the preceding paragraph may be at least 98 mol % and less than or equal to 100% of the total moles of organic components of the product composition in stream 128 from the second reactor 126, while the combined amount of other components including undesired byproducts (e.g., HFC-281) may be greater than or equal to 0 mol % and less than 2 mol % of the total moles of organic components of the product composition in stream 128 from the second reactor 126.

### Step (ii)-(a) Post Processing

Referring again to FIG. 1, the product stream 128 from second reactor 126 may go through one or more post processing steps for separating desired end product HFO-1252zc with recyclable intermediates (e.g., HFC-263fb) and/or undesired byproducts (e.g., HFC-281) to obtain and end product composition containing substantially pure HFO-1252zc. For example, product stream 128 may go through a caustic scrubber, a dryer column, and then to a fourth distillation column 130 to obtain an end product composition containing at least 99% pure HFO-1252zc.

For a dehydrofluorination reaction of Step (ii)-(a) carried out using each catalyst/support combination (each row) in Table 6, referring to FIG. 1, the amount of desired end product HFO-1252zc in top stream 132 may be greater than 90 mol %, 95 mol %, greater than 98 mol %, greater than 99 mol %, or greater than 99.9 mol %, and for each of the foregoing, less than or equal to 100 mol %, for example, based on total moles of organic components in the composition.

For a dehydrofluorination reaction of Step (ii)-(a) carried out using each catalyst/support combination (each row) in Table 6, referring to FIG. 1, the amount of unreacted raw material HFC-263fb in bottom stream 134 may be greater than 90 mol %, greater than 95 mol %, greater than 98 mol %, or greater than 99 mol %, and for each of the foregoing, less than or equal to 100 mol %, for example, based on total moles of organic components in the composition.

For a dehydrofluorination reaction of Step (ii)-(a) carried out using each catalyst/support combination (each row) in Table 6, referring to FIG. 1, the amount of undesired byproduct HFC-281 in bottom stream 134, if present, may be greater than or equal to 0.01 mol % and yet less than 5 mol %, less than 3 mol %, or less than 1 mol %, for example, based on total moles of organic components of the composition.

The bottom stream 134 containing mainly HFC-263fb may be recycled to second reactor 126 for dehydrofluorination to HFO-1252zc.

### VI. Step (ii)-(b)

In another example, dehydrofluorination reaction of Step (ii) is carried out in a suitable liquid phase reactor (e.g., reactor 126 as shown in FIG. 1). Step (ii)-(b) of Process 1 includes dehydrofluorinating HFC-263fb in liquid phase to produce HFO-1252zc. The liquid phase reactants and process conditions used in Step (ii)-(b) liquid phase dehydrofluorination reaction of Process 1 is detailed below.

### Step (ii)-(b) Caustic Solution

The dehydrofluorination reaction of Step (ii)-(b) may be carried out in a liquid phase reactor 126 in the presence of a caustic solution. Suitable bases that may be used to make the caustic solution include, but are not limited to, potassium hydroxide (KOH), sodium hydroxide (NaOH), calcium oxide (CaO), and/or calcium hydroxide (Ca(OH)₂).

Further to the foregoing, the hydrofluorination reaction of Step (ii)-(b) may be carried out in a protic solvent, such as water.

The caustic solution containing feed stream in reactor 126 can be an aqueous mixture, and can contain the base at any suitable strength for producing the desired reaction. For example, when KOH is used as the base, the base containing feed stream can contain base at a strength from about 1 wt. % to about 60 wt. %, from about 5 wt. % to about 55 wt. %, preferably from about 10 wt. % to about 50 wt. %, more preferably from about 40 wt. % to about 50 wt. %.

### Step (ii)-(b) Optional Catalyst

The dehydrofluorination reaction of Step (ii)-(b) may be carried out in a liquid phase reactor 126 optionally in the presence of a phase transfer agent or catalyst for enhancing the reaction rate. Suitable phase transfer catalyst include, but are not limited to, behentrimonium chloride (C₂₅H₅₄ClN) (e.g., Aliquat®336). Other phase transfer catalysts may be selected from polyethylene glycol, benzyltriethylammonium chloride, tetrabutylammonium bromide, tetrabutylammonium chloride, tetrabutylammonium bisulfate, trioctylmethyl ammonium chloride or hexamethylene-1-n-hexyldimethyl-6-octadecyldimethylammonium bromide.

### Step (ii)-(b) Reaction Temperature

For a liquid phase dehydrofluorination reaction of Step (ii)-(b) of Process 1 using bases including KOH, NaOH, CaO, and/or Ca(OH)₂ as described above, the reaction temperature of Step (ii)-(b) may be as low as about 40° C., about 45° C., about 50° C., or as high as about 100° C., about 110° C., about 120° C., about 130° C., about 140° C., about 150° C., or within any range encompassed by two of the foregoing values as endpoints, such as from about 50° C. to about 155° C., or from about 70° C. to about 130° C., for example. Specific examples of additional suitable ranges are set forth below in Table 9. The numerical ranges set forth in Table 9 below are understood to be prefaced by “about”.

TABLE 9
Temperature Ranges - Step (ii)-(b) Liquid
Phase Dehydrofluorination Reaction

| From (° C.) | To (° C.) |
|---|---|
| 40 | 150 |
| 40 | 140 |
| 40 | 130 |
| 40 | 120 |
| 40 | 110 |
| 40 | 100 |
| 50 | 150 |
| 50 | 140 |
| 50 | 130 |
| 50 | 120 |
| 50 | 110 |
| 50 | 100 |
| 70 | 150 |
| 70 | 140 |
| 70 | 130 |
| 70 | 120 |
| 70 | 110 |
| 70 | 100 |
| 100 | 150 |
| 100 | 140 |
| 100 | 130 |
| 100 | 120 |
| 100 | 110 |

### Step (ii)-(b) Reaction Conditions

A summary of the preferred caustic solution, catalyst, and temperature as discussed above are summarized in Table 10 below. The numerical ranges set forth in Table 10 are understood to be prefaced by “about”.

TABLE 10
Summary of Preferred Step (ii)-b
Liquid Phase Reaction Conditions

| Caustic Solution | Catalyst | Temperature (° C.) |
|---|---|---|
| KOH | None | 40-150 |
| KOH | None | 40-120 |
| KOH | None | 50-150 |
| KOH | None | 50-120 |
| KOH | None | 100-150 |
| KOH | None | 100-120 |
| KOH | behentrimonium chloride | 40-150 |
| KOH | behentrimonium chloride | 40-120 |
| KOH | behentrimonium chloride | 50-150 |
| KOH | behentrimonium chloride | 50-120 |
| KOH | behentrimonium chloride | 100-150 |
| KOH | behentrimonium chloride | 100-120 |

### Step (ii)-(b) Product Composition

For a liquid phase dehydrofluorination reaction of Step (ii)-(b) of Process 1 carried out using caustic solutions made with bases including KOH, NaOH, CaO, and/or Ca(OH)₂ as described above, referring to FIG. 1, the amount of HFO-1252zc in the product stream 128 from second reactor 126 may be at least 15 mol %, at least 25 mol %, at least 30 mol %, or at least 35 mol %, and for each of the foregoing, less than or equal to 100 mol %, for example, based on total moles of organic components of the composition.

For a liquid phase dehydrofluorination reaction of Step (ii)-(b) of Process 1 carried out using caustic solutions made with bases including KOH, NaOH, CaO, and/or Ca(OH)₂ as described above, referring to FIG. 1, the amount of HFC-263fb in the product composition in stream 128 from the second reactor 126 may be greater than or equal to 0.1 mol % and yet less than 85 mol %, less than 75 mol %, less than 70 mol %, or less than 65 mol %, for example, based on total moles of organic components of the composition.

For a liquid phase dehydrofluorination reaction of Step (ii)-(b) of Process 1 carried out using caustic solutions made with bases including KOH, NaOH, CaO, and/or Ca(OH)₂ as described above, referring to FIG. 1, the amount of undesired byproduct HFC-281 in the product composition in stream 128 from the second reactor 126, if present, may be greater than or equal to 0.1 mol % and yet less than 5 mol %, less than 4 mol %, or less than 2 mol %, for example, based on total moles of organic components of the composition.

For example, for a dehydrofluorination reaction of Step (ii)-(b) carried out using caustic solutions made with bases including KOH, NaOH, CaO, and/or Ca(OH)₂ as described above, referring to FIG. 1, in the product composition in stream 128 from the second reactor 126, the amount of HFO-1252zc may be at least 20 mol % and less than or equal to 99.9 mol %, the amount of HFC-263fb may be at least 0.1 mol % and less than or equal to 80 mol %, based on the combined total moles of the HFO-1252zc and HFC-263fb in the product composition.

The combined amount of HFO-1252zc and HFC-263fb in the composition set forth in the preceding paragraph may be at least 90 mol % and less than or equal to 100% of the total moles of organic components of the product composition in stream 128 from the second reactor 126, while the combined amount of other components including undesired byproducts (e.g., HFC-281) may be greater than or equal to 0 mol % and less than 10 mol % of the total moles of organic components of the product composition in stream 128 from the second reactor 126.

For example, for a dehydrofluorination reaction of Step (ii)-(b) carried out using caustic solutions made with bases including KOH, NaOH, CaO, and/or Ca(OH)₂ as described above, referring to FIG. 1, in the product composition in stream 128 from the second reactor 126, the amount of HFO-1252zc may be at least 30 mol % and less than or equal to 99.9 mol %, the amount of HFC-263fb may be at least 0.1 mol % and less than or equal to 70 mol %, based on the combined total moles of the HFO-1252zc and HFC-263fb in the product composition.

The combined amount of HFO-1252zc and HFC-263fb in the composition set forth in the preceding paragraph may be at least 95 mol % and less than or equal to 100% of the total moles of organic components of the product composition in stream 128 from the second reactor 126, while the combined amount of other components including undesired byproducts (e.g., HFC-281) may be greater than or equal to 0 mol % and less than 5 mol % of the total moles of organic components of the product composition in stream 128 from the second reactor 126.

For example, for a dehydrofluorination reaction of Step (ii)-(b) carried out using caustic solutions made with bases including KOH, NaOH, CaO, and/or Ca(OH)₂ as described above, referring to FIG. 1, in the product composition in stream 128 from the second reactor 126, the amount of HFO-1252zc may be at least 35 mol % and less than or equal to 99.9 mol %, the amount of HFC-263fb may be at least 0.1 mol % and less than or equal to 65 mol %, based on the combined total moles of the HFO-1252zc and HFC-263fb in the product composition.

The combined amount of HFO-1252zc and HFC-263fb in the composition set forth in the preceding paragraph may be at least 98 mol % and less than or equal to 100% of the total moles of organic components of the product composition in stream 128 from the second reactor 126, while the combined amount of other components including undesired byproducts (e.g., HFC-281) may be greater than or equal to 0 mol % and less than 2 mol % of the total moles of organic components of the product composition in stream 128 from the second reactor 126.

### Step (ii)-(b) Post Processing

Referring again to FIG. 1, the product stream 128 from second reactor 126 may go through one or more post processing steps for separating desired end product HFO-1252zc with recyclable intermediates (e.g., HFC-263fb) and/or undesired byproducts (e.g., HFC-281) to obtain an end product composition containing substantially pure HFO-1252zc. For example, product stream 128 may go through a drying column, and then to a fourth distillation column 130 to obtain an end product composition containing at least 99% pure HFO-1252zc.

For a liquid phase dehydrofluorination reaction of Step (ii)-(b) carried out using caustic solutions made with bases including KOH, NaOH, CaO, and/or Ca(OH)₂ as described above, referring to FIG. 1, the amount of desired end product HFO-1252zc in top stream 132 may be greater than 90 mol %, greater than 95 mol %, greater than 98 mol %, greater than 99 mol %, or greater than 99.9 mol %, and for each of the foregoing, less than or equal to 100 mol %, for example, based on total moles of organic components in the composition.

For a liquid phase dehydrofluorination reaction of Step (ii)-(b) carried out using caustic solutions made with bases including KOH, NaOH, CaO, and/or Ca(OH)₂ as described above, referring to FIG. 1, the amount of unreacted raw material HFC-263fb in bottom stream 134 may be greater than 90 mol %, greater than 95 mol %, greater than 98 mol %, or greater than 99 mol %, and for each of the foregoing, less than or equal to 100 mol %, for example, based on total moles of organic components in the composition.

For a liquid phase dehydrofluorination reaction of Step (ii)-(b) carried out using caustic solutions made with bases including KOH, NaOH, CaO, and/or Ca(OH)₂ as described above, referring to FIG. 1, the amount of undesired byproduct HFC-281 in bottom stream 134, if present, may be greater than or equal to 0.1 mol % and yet less than 5 mol %, less than 3 mol %, or less than 1 mol %, for example, based on total moles of organic components of the composition.

The bottom stream 134 containing mainly HFC-263fb may be recycled to second reactor 126 for dehydrofluorination to HFO-1252zc.

### EXAMPLES

### Example 1

### HCFO-1233zd Hydrogenation to HFC-263fb Using 0.2% Pd/Alpha(α)-Al₂O₃Catalyst

Example 1 shows conversion of HCFO-1233zd to HFC-263fb using H₂ and 0.2% Pd/alpha(α)-Al₂O₃ catalyst. The surface area of the Pd/alpha(α)-Al₂O₃ catalyst used is 2.6 m²/g.

The experimental apparatus used for this example includes a feed system containing gas flow controllers for N₂ and H₂ and a Micromotion mass flow meter connected to a research control valve (RCV) controlling the organic flow rate. The reactor consists of a one-inch SS tube packed with 20 ml of the catalyst diluted with 30 ml of SS Propack mesh (⅛″). The reactor is heated by placing it inside a box oven. A thermocouple is inserted into the middle of the catalyst bed to read the operating temperature. The pressure control system consists of a RCV which is controlling the pressure by getting feedback from the pressure transducer placed after the reactor. For GC analysis, samples are taken after the reactor using a Tedlar™ gas sampling bag filled with 50 ml of water to capture HCl and HF. Before the GC analysis, the sample bag is heated at 60° C. for at least 30 minutes to assure that all the organic content is in the gas phase. Then, a sample is taken using a syringe and injected into the GC instrument for analysis.

Prior to the reactions, the catalyst is purged with nitrogen and then is pretreated under 100 ml/min of hydrogen (H₂) at 200° C. for eight hours. Then, temperature is reduced to 130° C. under flowing hydrogen. When temperature is stable at 130° C., pressure is adjusted to 45 psig. After pressure is stable at about 45 psig, 10 g/h of HCFO-1233zd is added to the feed stream. Temperature increases to a range including but not limited to about 15 to 20° C. upon introduction of HCFO-1233zd; the temperature is adjusted to about 150° C. by changing the box oven temperature.

Product composition is determined using GC-FID analysis. The substrate conversion is 100%. Product stream comprises of about 89 mol % HFC-263fb (CH₃—CH₂—CF₃), 6 mol % HCFC-253 isomers (CH₂Cl—CH₂—CF₃ and CH₃—CHCl—CF₃), 1 mol % HFO-1243zf (CH₂═CH—CF₃), 2 mol % HFC-272fb (CH₃—CH₂—CHF₂), 1 mol % propane, and 1 mol % others. The recyclable intermediates are HCFC-253 isomers and HFO-1243zf. The major byproduct is HFC-272fb.

### Example 2

### HCFO-1233zd Hydrogenation to HFC-263fb Using 4% Pd/C Catalyst

Example 2 shows conversion of HCFO-1233zd to HFC-263fb using H₂ and 4% Pd/C catalyst. The surface area of the Pd/C catalyst used is around 1500 m²/g.

The experimental apparatus and methods used for this example are similar to Example 1. The reactor consists of a one-inch SS tube packed with 70 ml of the catalyst. Prior to the reactions, the reactor is purged with nitrogen and then the catalyst is pretreated under 300 ml/min of hydrogen (H₂) at 280° C. for eight hours. Then, temperature is reduced to 200° C. under flowing hydrogen. When temperature is stable at 200° C., pressure is adjusted to 45 psig. After pressure is stable at about 45 psig, 30 g/h of HCFO-1233zd is added to the feed stream. Temperature increases to a range including but not limited to about 40 to 50° C. upon introduction of HCFO-1233zd; the temperature is adjusted to about 280° C. by changing the box oven temperature.

Product composition is determined using GC-FID analysis. The substrate conversion is 100%. Product stream comprises of about 91 mol % HFC-263fb (CH₃—CH₂—CF₃), 1 mol % HCFC-253 isomers (CH₂Cl—CH₂—CF₃ and CH₃—CHCl—CF₃), 3 mol % HFC-272fb (CH₃—CH₂—CHF₂), 3 mol % propane, 1 mol % HFO-1243zf (CH₂═CH—CF₃), 0.5 mol % HFC-281(CH₃—CH₂—CH₂F), and 0.5 mol % others. The recyclable intermediates are HCFC-253 isomers and HFO-1243zf. The major byproducts are HFC-272fb, HFC-281, and propane.

### Example 3

### Liquid Phase Conversion of HFC-263fb Using KOH

This example shows liquid phase conversion of HFC-263fb to HFO-1252zc.

The experimental setup includes a 2-gallon Hastelloy C pressure vessel (Parr Instruments). The vessel is equipped with an agitator. The exit of reactor goes into distillation column (1″ OD, 18″ length). The product of the distillation goes into product collection cylinder (PCC). Reaction is performed in batch mode. The reactor is loaded with 1790 grams of organic feed. Organic feed consisted of about 99.8 GC wt. % of HFC-263fb and 0.2 wt. % 1-fluoropropane (HFC-281).

The reactor is heated to 115° C. with stirrer (600 rpm) on. When the reactor is at the desired temperature and temperature is stable, 2430 g of 45% KOH solution is added over three hours using Eldex pump. After complete addition of KOH solution, the reaction mixture is kept at 115° C. under stirring for an additional 0.5 hours. Then the reactor exit is opened to distillation column. Due to some fluctuations in temperature, the system is allowed to equilibrate for 2 hours (total reflux). Then the hold time of 10 hours is started. At the end of the hold time, the reactor temperature is reduced to 70° C. and the reactor is opened to a dry-ice cooled product collection cylinder (PCC).

About 1587 g of organic product is collected in the PCC. The overall mass balance is 96%. The organic product is analyzed using gas chromatography. The mol % of 1252zc and HFC-263fb are 32 and 67, respectively.

### Example 4

### Liquid Phase Conversion of HFC-263fb Using KOH

This example shows liquid phase conversion of HFC-263fb to HFO-1252zc.

The experimental setup includes a 2-gallon Hastelloy C pressure vessel (Parr Instruments). The vessel is equipped with agitator. The exit of reactor goes into distillation column (1″ OD, 18″ length). The product of the distillation goes into product collection cylinder (PCC). Reaction is performed in batch mode. Reactor is loaded with 1792 grams of organic feed and 6 g of Aliquat® phase transfer catalyst. Organic feed consisted of about 99.5 GC wt. % of HFC-263fb and about 0.5 GC wt. % of HFC-281.

The reactor is heated to 115° C. with stirrer (600 rpm) on. When the reactor is at the desired temperature and temperature is stable, 2450 g of 45% KOH solution is added over three hours using Eldex pump. After complete addition of KOH solution, the reaction mixture is kept at 115° C. under stirring for an additional 0.5 hours. Then the reactor exit is opened to distillation column. Due to some fluctuations in temperature, the system is allowed to equilibrate for 2 hours (total reflux). Then the hold time of 10 hours is started. At the end of the hold time, the reactor temperature is reduced to 70° C. and the reactor is opened to a dry-ice cooled product collection cylinder (PCC).

About 1590 g of organic products are collected in the PCC. The overall mass balance is 98.1%. The organic product is analyzed using gas chromatography. The mol % of HFO-1252zc, HFC-263fb, and HFC-281 are 36.9, 61.6, and 1.5%, respectively.

### Example 5

### Integrated Process for Producing HFO-1252zc from HCFO-1233zd

An example of Integrated process for conversion of HCFO-1233zd to HFO-1252zc is shown in FIG. 1. In the first step, HFO-1233zd is hydrogenated to HFC-263fb in reactor 104 using H₂ and a hydrogenation catalyst such as Pd on carbon or alumina. See Examples 1-2 for the reaction conditions and product compositions.

The reactor effluent from reactor 104 is sent to a scrubber, and then to a dryer tube, and then to first distillation column 108. The top stream from first distillation column 108 comprises propane and HFO-1243zf. This stream could be recycled back to reactor 104 for HFO-1243zf hydrogenation. However, occasional vent is necessary to remove propane from the system. The bottom stream from first distillation column 108 comprises of HFC-263fb, HFC-272fb, HFC-281, and HCFC-253 isomers (HCFC-253db and HCFC-253fb), and is sent to second distillation column 114.

The top stream from second distillation column 114 comprises of HFC-263fb and HFC-281, and is sent to second reactor 126. The bottom stream from second distillation column 114 comprises of HFC-272fb and HCFC-253 isomers, and can be optionally sent to third distillation column 118 to remove HFC-272fb from the top stream. HCFC-253 isomers are considered recyclable intermediates and can optionally be sent back to first reactor 104 for their conversion to HFC-263fb.

The top stream from second distillation column 114 is sent to second reactor 126. See Examples 3-4 for the reaction conditions and product compositions. The effluent stream from second reactor 126 comprises of HFO-1252zc, HFC-263fb, and HFC-281, and is sent to fourth distillation column 130 for the separation of HFO-1252zc product from unreacted HFC-263fb.

The top stream from fourth distillation column 130 comprises of about 99.9% pure HFC-1252zc final product. The bottom stream from fourth distillation column 130 comprises of HFC-263fb and HFC-281, and is collected or sent back to second reactor 126. However, occasional venting of this stream is required to prevent HFC-281 accumulation in the system.

### Example 6

### Integrated Process for Producing HFO-1252zc from HCFO-1233zd

HCFO-1233zd is hydrogenated to HFC-263fb in a fixed bed tubular reactor using Pd/C catalyst with around 1500 m²/g surface area.

The first reactor 104 consists of a bundle of one-inch SS packed bed reactors with the total volume of 70 liters. The reactor heating and cooling is done using a jacket of circulating hot oil. Prior to the reactions, the first reactor 104 is purged with nitrogen and then the catalyst is pretreated under 300 lit/min of hydrogen (H₂) at 280° C. for eight hours. Then, temperature is reduced to 200° C. under flowing hydrogen. When temperature is stable at 200° C., 30 kg/h of HCFO-1233zd is added to the feed stream. Then, the temperature is adjusted to about 280° C. at the hot zone. The contact time is about 20-22 seconds and hydrogen/HCFO-1233zd ratio is about 3.5.

Under these conditions, HCFO-1233zd conversion is 100% and the reactor effluent comprises of 87 mol % CH₃—CH₂—CF₃ (HFC-263fb), 4 mol % HCFC-253 isomers (CH₂Cl—CH₂—CF₃ and CH₃—CHCl—CF₃), 3 mol % HFC-272fb (CH₃—CH₂—CHF₂), 3 mol % propane, 2 mol % HFO-1243zf (CH₂═CH—CF₃), 0.5 mol % HFC-281 (CH₃—CH₂—CH₂F), and 0.5 mol % others. The recyclable intermediates are HCFC-253 isomers and HFO-1243zf. The major byproducts are HFC-272fb, HFC-281, and propane.

The reactor effluent from first reactor 104 is sent to a scrubber, and then to a dryer tube, and then to first distillation column 108. The top stream from first distillation column 108 comprises about 60 mol % propane and 40 mol % HFO-1243zf. This stream could be optionally recycled back to first reactor 104 for HFO-1243zf hydrogenation. However, occasional vent is necessary to remove propane from the system. The bottom stream from first distillation column 108 comprises of about 91.6 mol % HFC-263fb, 2 mol % HFC-272fb, 0.5 mol % HFC-281, and 2 mol % HCFC-253 isomers (HCFC-253db and HCFC-253fb) and is sent to second distillation column 114. The top stream from second distillation column 114 comprises of about 99.4 mol % HFC-263fb and 0.6 mol % HFC-281, and is sent to second reactor 126. The bottom stream from second distillation column 114 comprises of about 40.0 mol % HFC-272fb and 53.3 mol % HCFC-253 isomers, and 6.7 mol % others, and is optionally sent to third distillation column 118 to remove HFC-272fb from the top stream. HCFC-253 isomers are considered recyclable intermediates and can optionally be sent back to first reactor 104 for their conversion to HFC-263fb.

The top stream from second distillation column 114 is collected and then is sent to second reactor 126. Second reactor 126 is a 300-gallon Inconel 600 stirred tank reactor with a distillation column on its top. The reaction is performed in semi-continuous mode. First, 390 kg of the organic from the top stream of second distillation column 114 is added to the second reactor 126. Then, about 1.2 kg of Aliquat® phase transfer catalyst is added to the second reactor 126. Second reactor 126 is heated under stirring to 115° C.

Once the temperature is stable, 490 kg of 45% KOH solution is slowly added over three hours while stirring. After complete addition of KOH solution, the reaction mixture is kept at 115° C. under stirring for an additional 0.5 hours. Then the reactor exit is opened to the distillation column. Due to some fluctuations in temperature, the system is allowed to equilibrate for 2 hours (total reflux). Then the hold time of 10 hours is started. At the end of the hold time, the reactor temperature is reduced to 70° C. and the reactor is opened to a chilled product collection cylinder. The reaction product from second reactor 126 comprises of about 37.3 mol % HFO-1252zc, 62.1 mol % HFC-263fb, and 0.6 mol % HFC-281. This product is sent to fourth distillation column 130 for the separation of HFO-1252zc product from unreacted HFC-263fb.

The top stream from fourth distillation column 130 comprises of about 99.9 mol % HFC-1252zc. The bottom stream from fourth distillation column 130 comprises of about 99.1 mol % HFC-263fb and 0.9 mol % HFC-281 and is collected or sent back to second reactor 126.

### ASPECTS

Aspect 1 is a method for producing 1,1-difluoropropene (HFO-1252zc), comprising reacting 1-chloro-3,3,3-trifluoropropene (HCFO-1233zd) with hydrogen in the presence of a catalyst to produce a first product composition comprising 1,1,1-trifluoropropane (HFC-263fb); and reacting HFC-263fb from the first product composition to produce a second product composition comprising 1,1-difluoropropene (HFO-1252zc).

Aspect 2 is the method of Aspect 1, wherein the first reacting step is conducted at a temperature of from about 100° C. to about 300° C.

Aspect 3 is the method of Aspect 1 or Aspect 2, wherein the catalyst is palladium metal supported on a carbon support.

Aspect 4 is the method of Aspect 1 or Aspect 2, wherein the catalyst is palladium metal supported on an alpha aluminum support.

Aspect 5 is the method of any one of Aspects 1 to 4, wherein the first product composition comprises 85 mol % to 99.8 mol % 1,1,1-trifluoropropane (HFC-263fb); 0.01 mol % to 10 mol % chlorotrifluoropropane isomers (HCFC-253 isomers); and 0.01 mol % to 5 mol % 3,3,3-trifluoropropene (HFO-1243zf), based on the combined total moles of the HFC-263fb, HCFC-253 isomers, and HFO-1243zf in the first product composition.

Aspect 6 is the method of any of Aspects 1 to 5, wherein the first product composition comprises a total amount of 1,1,1-trifluoropropane (HFC-263fb), chlorotrifluoropropane isomers (HCFC-253 isomers), and 3,3,3-trifluoropropene (HFO-1243zf) of at least 85 mol %, based on total moles of organic components of the first product composition.

Aspect 7 is the method of any one of Aspects 1 to 6, further comprising distilling the first product composition to produce a distilled composition comprising at least 90 mol % HFC-263fb.

Aspect 8 is the method of any one of Aspects 1 to 7, wherein the second reacting step is conducted in vapor phase in the presence of a second catalyst at a second temperature.

Aspect 9 is the method of Aspect 8, wherein the second catalyst comprises at least one of chromium oxide (Cr₂O₃), zinc oxide (ZnO), nickel oxide (NiO), aluminum oxide (Al₂O₃), iron oxide (Fe₂O₃), and magnesium oxide (MgO), chromium fluoride (CrF₃), alumina fluoride (AlF₃), iron fluoride (FeF₃), magnesium fluoride (MgF₂), and combinations of the foregoing.

Aspect 10 is the method of Aspect 8 or Aspect 9, wherein the second temperature is from about 125° C. to about 500° C.

Aspect 11 is the method of any one of Aspects 8 to 10, wherein the second product composition comprises 15 mol % to 99.9 mol % 1,1-difluoropropene (HFO-1252zc); and 0.01 mol % to 85 mol % 1,1,1-trifluoropropane (HFC-263fb), based on the combined total moles of the HFC-263fb and HFO-1252zc in the second product composition.

Aspect 12 is the method of Aspect 11, wherein the second product composition comprises a total amount of 1,1-difluoropropene (HFO-1252zc) and 1,1,1-trifluoropropane (HFC-263fb) of at least 90 mol %, based on total moles of organic components of the second product composition.

Aspect 13 is the method of any one of Aspects 8 to 12, further comprising distilling the second product composition to produce an end product composition comprising at least 99 mol % HFO-1252zc.

Aspect 14 is the method of any one of Aspects 1 to 7, wherein the second reacting step comprises reacting HFC-263fb from the first product composition in the presence of a caustic solution to produce the second product composition.

Aspect 15 is the method of Aspect 14, wherein at least one of the following conditions is present: the caustic solution comprises a protic solvent; and the caustic solution is formed from a base comprising at least one of KOH, NaOH, CaO, Ca(OH)₂, and combinations of the foregoing.

Aspect 16 is the method of Aspect 14 or Aspect 15, wherein the second reacting step is conducted at a second temperature of from about 40° C. to about 150° C.

Aspect 17 is the method of any one of Aspects 14 to 16, wherein the second reacting step is conducted in the presence of a phase transfer catalyst.

Aspect 18 is the method of Aspect 17, wherein the phase transfer catalyst comprises behentrimonium chloride.

Aspect 19 is the method of any one of Aspects 14 to 18, wherein the second product composition comprises 15 mol % to 99.9 mol % 1,1-difluoropropene (HFO-1252zc); and 0.01 mol % to 85 mol % 1,1,1-trifluoropropane (HFC-263fb), based on the combined total moles of the HFC-263fb and HFO-1252zc in the second product composition.

Aspect 20 is the method of Aspect 19, wherein the second product composition comprises a total amount of 1,1-difluoropropene (HFO-1252zc) and 1,1,1-trifluoropropane (HFC-263fb) of at least 90 mol %, based on total moles of organic components of the second product composition.

Aspect 21 is the method of any one of Aspects 14 to 20, further comprising distilling the second product composition to produce an end product composition comprising at least 99 mol % HFO-1252zc.

Aspect 22 is a product composition comprising 1,1-difluoropropene (HFO-1252zc) made according to any one of Aspects 1 to 21.

What is claimed is:

1. A method for producing 1,1-difluoropropene (HFO-1252zc), comprising:
reacting 1-chloro-3,3,3-trifluoropropene (HCFO-1233zd) with hydrogen in the presence of a catalyst to produce a first product composition comprising 1,1,1-trifluoropropane (HFC-263fb); and
reacting HFC-263fb from the first product composition to produce a second product composition comprising 1,1-difluoropropene (HFO-1252zc).

2. The method of claim 1, wherein the first reacting step is conducted at a temperature of from about 100° C. to about 300° C.

3. The method of claim 1, wherein the catalyst is palladium metal supported on one of a carbon support and an alpha aluminum support.

4. The method of claim 1, wherein the first product composition comprises:
85 mol % to 99.8 mol % 1,1,1-trifluoropropane (HFC-263fb);
0.01 mol % to 10 mol % chlorotrifluoropropane isomers (HCFC-253 isomers); and
0.01 mol % to 5 mol % 3,3,3-trifluoropropene (HFO-1243zf), based on the combined total moles of the HFC-263fb, HCFC-253 isomers, and HFO-1243zf in the first product composition.

5. The method of claim 4, wherein the first product composition comprises a total amount of 1,1,1-trifluoropropane (HFC-263fb), chlorotrifluoropropane isomers (HCFC-253 isomers), and 3,3,3-trifluoropropene (HFO-1243zf) of at least 85 mol %, based on total moles of organic components of the first product composition.

6. The method of claim 1, further comprising:
distilling the first product composition to produce a distilled composition comprising at least 90 mol % HFC-263fb.

7. The method of claim 1, wherein the second reacting step is conducted in vapor phase in the presence of a second catalyst at a second temperature.

8. The method of claim 7, wherein the second catalyst comprises at least one of chromium oxide (Cr₂O₃), zinc oxide (ZnO), nickel oxide (NiO), aluminum oxide (Al₂O₃), iron oxide (Fe₂O₃), and magnesium oxide (MgO), chromium fluoride (CrF₃), alumina fluoride (AlF₃), iron fluoride (FeF₃), magnesium fluoride (MgF₂), and combinations of the foregoing.

9. The method of claim 7, wherein the second temperature is from about 125° C. to about 500° C.

10. The method of claim 7, wherein the second product composition comprises:
15 mol % to 99.9 mol % 1,1-difluoropropene (HFO-1252zc); and
0.01 mol % to 85 mol % 1,1,1-trifluoropropane (HFC-263fb), based on the combined total moles of the HFC-263fb and HFO-1252zc in the second product composition.

11. The method of claim 10, wherein the second product composition comprises a total amount of 1,1-difluoropropene (HFO-1252zc) and 1,1,1-trifluoropropane (HFC-263fb) of at least 90 mol %, based on total moles of organic components of the second product composition.

12. The method of claim 7, further comprising:
distilling the second product composition to produce an end product composition comprising at least 99 mol % HFO-1252zc.

13. The method of claim 1, wherein the second reacting step comprises reacting HFC-263fb from the first product composition in the presence of a caustic solution to produce the second product composition.

14. The method of claim 13, wherein at least one of the following conditions is present:
the caustic solution comprises a protic solvent; and
the caustic solution is formed from a base comprising at least one of KOH, NaOH, CaO, Ca(OH)₂, and combinations of the foregoing.

15. The method of claim 13, wherein the second reacting step is conducted at a second temperature of from about 40° C. to about 150° C.

16. The method of claim 13, wherein the second reacting step is conducted in the presence of a phase transfer catalyst, the phase transfer catalyst comprising behentrimonium chloride.

17. The method of claim 13, wherein the second product composition comprises:
15 mol % to 99.9 mol % 1,1-difluoropropene (HFO-1252zc); and
0.01 mol % to 85 mol % 1,1,1-trifluoropropane (HFC-263fb), based on the combined total moles of the HFC-263fb and HFO-1252zc in the second product composition.

18. The method of claim 17, wherein the second product composition comprises a total amount of 1,1-difluoropropene (HFO-1252zc) and 1,1,1-trifluoropropane (HFC-263fb) of at least 90 mol %, based on total moles of organic components of the second product composition.

19. The method of claim 13, further comprising:
distilling the second product composition to produce an end product composition comprising at least 99 mol % HFO-1252zc.

20. A product composition comprising 1,1-difluoropropene (HFO-1252zc) made according to the method of claim 1.
